(19) United States
(12) Patent Application Publication
(10) Pub. No.: US 2008/0312331 A1
(43) Pub. Date: Dec. 18, 2008

(54) Method of Treating Dailysis-Induced Hypotension

(75) Inventor: Lawrence Solomon, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(73) Assignee: SOLAPHARM, INC., Plantation, FL (US)

(21) Appl. No.: 11/815,226

(22) PCT Filed: Feb. 3, 2006
(86) PCT No.: PCT/US06/03801

§ 371 Date: Aug. 1, 2007

Related U.S. Application Data
(60) Provisional application No. 60/649,808, filed on Feb. 3, 2005.

(51) Int. Cl. A61K 31/165

(52) U.S. Cl. 514/626

(57) ABSTRACT

A method is described for the treatment of hypotension, e.g., intra-dialytic hypotension, which is based on the administration to a patient, e.g. a dialysis patient during dialysis, of midodrine or a midodrine metabolite which will increase a lowered blood pressure in the patient or relieve a symptom caused by the dialysis procedure.

### FIELD OF THE INVENTION

The invention is directed to pharmacologic treatment of hypotensive or symptomatic complications of dialysis, especially in renal failure patients.

### BACKGROUND OF THE INVENTION

Limited attention has been paid to the development of safe and effective pharmaceutical methods of improving the procedure of dialysis. For example, the most common complication occurring during the procedure of hemodialysis is symptomatic drop in blood pressure (hypotension), which is generally treated by postural changes and intravenous fluid administration.

The syndrome of symptomatic severe IDH in chronic renal failure IDH patients appears to have multiple potential etiologies. Henrich et al., Kid. Inter. (1986) 30:605-612, which is incorporated by reference. To that end, a variety of treatments have been proposed, including but not limited to selective serotonin reuptake inhibitors such as sertraline and paroxetine (which are not known to have direct vasoconstrictive or anti-hypotensive effects), adenosine antagonism (such as via caffeine), and the use of cold dialysate fluid.

Certain patients are known to be prone to severe symptomatic intradialytic hypotension (“IDH”). Along with unpleasant symptoms, various serious complications of IDH have been well noted. See, e.g., Schreiber, Am. J Kidney Dis. (2001) 38(4):Suppl. 4 S1-S10.

It is known that patients suffering from chronic renal failure necessitating chronic hemodialysis treatments may be administered oral midodrine prior to the hemodialysis procedure. This drug has been suggested to benefit patients who suffer the following complications during the hemodialysis procedure:

1. Symptoms such as dizziness, diaphoresis, nausea, cramps, weakness, and blurred vision; and
2. Drop in systolic blood pressure (“SBP”) of at least 20 mm Hg relative to the BP before initiation of the hemodialysis procedure; and who suffer from the listed symptoms and/or SBP drop in at least 50% of all dialyses.

The above usage has not been systematically studied and has not been approved by the U.S. Food and drug Administration or, to the inventor's knowledge, any other regulatory agency as safe and effective for preventing or treating intradialytic hypotension, whether symptomatic or not. A review of the field is provided by Prakash et al., Nephrol. Dial. Transplant. (2004) 19:2553-2558. These authors call for a large-scale randomized study to confirm the safety and efficacy that they believe was suggested by the small-scale, mostly non-randomized studies of pre-dialysis use of oral midodrine in frequent sufferers of symptomatic severe hypotension during dialysis.

Typically, the hemodialysis procedure is undergone three times weekly, for a limited period of time, such as three hours, for each procedure. Midodrine, an alpha-adrenergic agonist, has been prophylactically administered per os, prior to hemodialysis, to prevent a hypotensive response in the hemodialysis patient. Peer et al., Nephrol. Dial. Transplant (2001) 16:1436-1441. In Flynn et al., Clin. Neph. 45(4) 261 (1996), the use of orally administered midodrine as a pre-dialysis medication is also disclosed. The dosing of midodrine has been disclosed to be 15-30 minutes before the start of a hemodialysis session, and such method of use has been limited to oral use and has been limited to hemodialysis in the chronic renal failure patient. No teaching has been identified that describes administering an anti-hypotensive agent to a patient undergoing dialysis for acute renal failure caused by, for example, exposure to a drug or from ingestion of or exposure to a toxic substance or poison.

When orally administered, there is a time lag between first absorption of midodrine into the body, then a further time lag to peak midodrine blood levels, then a further time lag to peak levels of an active metabolite known as desglymidodrine, to which the major pharmacologic effects of midodrine are ascribed. Midodrine is considered to be a prodrug. Desglymidodrine is removed by hemodialysis and is predominantly cleared renally in patients of normal renal function. Information about midodrine may be found in the Product Information for the ProAmatine® brand of midodrine marketed in the U.S., which is incorporated by reference, as described in the 2004 Physician's Desk Reference. Because of the above predictable sequence of events, the specific method of midodrine prophylactic administration that has been disclosed does not suggest other methods of administering midodrine. It has not heretofore been suggested to administer desglymidodrine for the purpose of either preventing or treating dialysis patients.

The specific procedure of hemodialysis for renal failure involves alteration of bodily physiology and, in the case of midodrine, affects the pharmacokinetics and likely the pharmacodynamics of the drug's effects, especially given the dialyzable nature of this drug and of desglymidodrine. In addition, the very act of initiating hemodialysis requires a physical procedure to gain vascular access to a shunt or graft generally within a person's body. It is thus reasonable to consider that a method of administration of a drug before the onset of this procedure is materially different from such administration subsequent to the initiation of the procedure.

The disclosed dosing of oral midodrine at an appropriate time before the onset of hemodialysis, which has not yet been demonstrated to be safe and effective for prevention of recurrent IDH in renal failure patients, is accordingly not predictive of success in either preventing or treating IDH when administering oral midodrine, or other forms of midodrine, during hemodialysis.

In comparison to midodrine that is swallowed, the very different pharmacokinetics of parenterally administered midodrine, including administration by transdermal or buccal routes (i.e., modes of administration other than per os), prior to hemodialysis, mean that any potential safe and effective use of pre-hemodialysis oral midodrine for renal hemodialysis patients would not predict such safe and effective use when given by these other modalities. For example, the oral prophylactic use of midodrine would not suggest its safe and effective use when administered by a route other than per os, e.g., parenteral administration such as intravenous (i.v.) or intramuscular (i.m.) administration or the like.

One of the concerns of such use would be safety, such as the concern that blood levels of midodrine peak prematurely to those produced by oral use of midodrine and could thereby adversely affect the cardiovascular function of the patient, such as by potentially inducing unwanted elevation of blood pressure. Another concern would be whether such use would be efficacious.

“Indications” that are “similar to midodrine” are also disclosed for a midodrine active metabolite, desglymidodrine, in U.S. Patent Application, Publication No. US 2002/0147232. However, such prophylactic use of orally administered midodrine, or its active metabolite, would not have been predictive of its use during dialysis as a treatment for intra- or post-dialytic hypotension, or symptoms such as nausea or vomiting, cramping, faintness, dizziness, weakness, fatigue, or diaphoresis associated with such a procedure, i.e., occurring in anticipation of, during, or after the procedure.

The prior art further fails to teach or suggest use of midodrine or any of its metabolites as an effective method of treating a patient to increase systolic blood pressure.

There is a present need for an effective treatment of intra-dialytic hypotension that occurs in patients undergoing dialysis. Most patients who exhibit intra-dialytic hypotension have essentially normal pre-dialysis systolic blood pressures, i.e. greater than or equal to 100 mm Hg in adults. There is also a need for a treatment while undergoing dialysis which can increase systolic blood pressure in a patient having decrease systolic pressure. The subject invention discloses novel methods of treating and preventing the serious and unpleasant syndrome of IDH and/or related signs and symptoms.

### SUMMARY OF THE INVENTION

The invention concerns, for example, a method of treating a patient experiencing or at risk of experiencing, certain signs or symptoms, for example, episodes of hypotension, nausea or vomiting, cramping, faintness, dizziness, weakness, fatigue, or diaphoresis, by administering an effective amount of an anti-hypotensive agent, preferably a vasoconstrictive agent or compound, e.g., an alpha-adrenergic agonist such as midodrine, or a pharmacologically effective metabolite thereof, to the patient.

One object of the invention is to provide a method for treating a patient requiring dialysis or an ultrafiltration procedure, e.g., a patient having renal failure, for a complication associated with that procedure. The patient may require dialysis for chronic renal failure or acute renal failure, such as caused by exposure to a drug or a toxic substance; acute myocardial infarction; or rhabdomyolysis. The method comprises administering during the dialysis or ultrafiltration procedure, an effective amount of a vasoconstrictive compound or agent, e.g., midodrine, or a salt, derivative, isomer, polymorph, or metabolite thereof, to treat or prevent a complication of said dialysis or ultrafiltration procedure. The renal failure may be chronic, as in end-stage renal disease, or may be acute.

Complications of the dialysis procedure for which the subject invention is intended to provide benefit include without limitation signs or symptoms such as hypotension, nausea or vomiting, cramping, weakness, faintness, fatigue, dizziness, or diaphoresis. For example, a patient suffering from renal failure requiring hemodialysis may be administered per the invention an effective amount of midodrine during the hemodialysis procedure in order to ameliorate or reverse a hypotensive episode occurring during the procedure.

For purposes of the subject invention, “hypotension” includes absolute hypotension as may be defined medically (such as SBP below 90 mm Hg), as well as relative hypotension, which is any lowered blood pressure in a patient relative to that patient's baseline blood pressure, whether or not that measurement falls within the medical definition of “hypotension.”

A primary object of the invention is to provide a method for the treatment of intra-dialytic hypotension. In a preferred embodiment, the subject invention comprises administering an effective amount of midodrine, or its active metabolite, desglymidodrine, to a patient following the onset of a hypotensive state or a symptom that is typically associated with low blood pressure. More preferably, the subject invention concerns a method for treating with midodrine or its active metabolite, desglymidodrine, a dialysis patient, during dialysis, in response to a hypotensive state induced during dialysis.

It is also an object of the invention to ameliorate one or all of the symptoms associated with intra-dialytic hypotension, e.g. nausea or vomiting, cramping, dizziness, weakness, faintness, fatigue, or diaphoresis, which may improve efficiency of dialysis (often measured by the formula KT/V).

It is also an object of the invention to provide a method of treating a hypotensive episode during dialysis that develops in a patient who was not previously diagnosed with hypotension or otherwise being currently treated with an anti-hypotensive agent, e.g., an alpha-adrenergic agonist for hypotension or a symptom associated with hypotension.

It is yet another object of the invention to provide a method of treating a sign or symptom, e.g., hypotension, nausea or vomiting, faintness, fatigue, weakness, diaphoresis or dizziness, before, during, or following a medical procedure that is associated with causing such symptoms. For example, a chronic renal failure patient may experience one or more of the symptoms of nausea, dizziness, or fainting during peritoneal dialysis. Therefore, the subject invention includes treatment as a pre-medication for conditions or symptoms associated with such medical procedures. Preferably, the subject invention concerns a method wherein the active agent is administered to a patient that is not under general anesthesia, i.e., anesthetized to an unconscious state. Typically, the patient is conscious when the subject treatment is administered, except following a fainting spell where consciousness may be temporarily lost.

These and other objects of the invention will become apparent from the appended specification.

### DETAILED DESCRIPTION OF THE INVENTION

One embodiment of the subject invention relates to a method of treating a patient to reverse or alleviate a hypotensive event associated with a medical procedure being administered to the patient. The subject method therefore comprises administering to the patient experiencing such a hypotensive event an effective amount of an active pharmaceutical ingredient (API) to alleviate or reverse the symptoms being exhibited by the patient.

The symptoms associated with a hypotensive event include without limitation one or more of nausea or vomiting, cramping, dizziness, diaphoresis, weakness, fatigue, and faintness. However, it should be understood that the subject method includes administering to a patient an effective amount of an API to alleviate one or more of the symptoms, whether or not the patient is hypotensive. Accordingly, the invention includes treating a patient for at least one of the above symptoms when these symptoms may arise from anticipation of, initiation of, or carrying out a medical procedure such as dialysis.

The method of the subject invention comprises administering an effective amount of an active pharmaceutical ingredient (API) to a patient to alleviate or reverse a particular symptom, such as nausea, dizziness, diaphoresis, fainting, hypotension, or the like, which is associated with, or in response to the medical procedure the patient is to undergo or is undergoing.

Preferably, the API administered to the patient in accordance with the subject invention is an anti-hypotensive agent, e.g., a vasoconstricting agent. More preferably, the API is an alpha-adrenergic agonist which has the property of raising blood pressure. Alpha-adrenergic agonists include, for example, epinephrine, norepinephrine, dobutamine, phenylephrine, albuterol, terbutaline, amphetamine, tyramine, methylnorepinephrine, ephedrine, and phenylpropanolamine. One preferred alpha-adrenergic agonist is midodrine. Midodrine is also known as 2-amino-N-[2,5-dimethoxyphenyl)-2-hydroxyethyl] acetamide. Midodrine may be made according to the procedures of U.S. Pat. No. 3,340,298. Midodrine is a prodrug and its principal active metabolite, desglymidodrine, may be used in the treatment method of the subject invention as an alternative to midodrine. One or more salts, derivatives, isomers, or polymorphs of midodrine, or one or more active metabolite of midodrine, e.g., desglymidodrine, may be used in accordance with the subject invention.

The API used in the method of the subject invention, e.g., midodrine, may for example be administered orally to be swallowed, by gastric feeding tube, buccally, transdermally, intravenously, intramuscularly, subcutaneously, or by suppository, or other convenient route. The preferred dose of the alpha-adrenergic agonist will be from 0.01 to 1 mg/kg of body weight per day, and may vary depending on the response of each individual patient.

The useful dosage forms include but are not limited to tablets, capsules, sterile isotonic solutions and transdermal patches which are made by conventional techniques such as those described in Remington, The Science and Practice of Pharmacy, 20th Ed. 2000, chapters 41, 42, 46 and 47, which are incorporated by reference. Oral tablets are available in the U.S. as ProAmatine in 2.5 mg, 5.0 mg, and 10.0 mg strengths.

It is believed that the use of midodrine or its metabolite, desglymidodrine has been disclosed only for prevention of intra-dialytic hypotension (IDH) as an orally administered dosage form. Accordingly, the subject invention includes the prevention of IDH by administering any vasoconstrictive agent, preferably an alpha-adrenergic agonist, and more preferably midodrine or desglymidodrine by an administration route other than per os. Thus the subject invention includes a method for prevention or treatment of IDH by administering a vasoconstrictive agent such as midodrine intravenously, intramuscularly, transdermally, rectally, or other non-oral or parenteral administration.

In common usage in the medical field, the term “hemodialysis” is often shortened to “dialysis.” In the present application, “dialysis” is used to mean “hemodialysis,” the dialytic process using blood as the substrate, and “peritoneal dialysis,” which involves dialysis of peritoneal fluid to remove one or more harmful or toxic substances therefrom. Dialysis is based on the diffusion of filterable substances across a semipermeable membrane and is also used in certain cases of acute drug intoxication. However, any reference to “dialysis” herein should be understood to include any procedure whereby body fluid is subjected to treatment, extra-corporeally (as in hemodialysis) or within the body (as in peritoneal dialysis) for purposes of eliminating a harmful substance from, or otherwise purifying or cleansing, that fluid, or to treat said fluid such as by subjecting it to ultraviolet light, incubating a pharmaceutical with the temporarily removed red or white blood cells, or the like, as medical technology may make advisable and practical. Therefore, the subject method is not limited to prevention or treatment of a patient undergoing “hemodialysis” or “peritoneal dialysis”. The term “dialysis” as used herein, including the claims attached hereto, includes a patient undergoing any dialysis procedure, or any ultrafiltration procedure, or any other procedure where a body fluid is treated as described, and such procedure may induce or be associated with a hypotensive effect or a symptom of hypotension may be exhibited by the patient.

The subject method therefore includes treating or preventing hypotension or a symptom associated with hypotension by administering a vasoconstrictor to a patient, in accordance with the subject invention, that may undergo a medical procedure which involves treating a body fluid extra-corporeally or within the body for purposes of modifying that body fluid. For example, the method includes treatment or prevention of hypotension or a symptom associated with hypotension by administering a vasoconstrictor before, during, or after peritoneal dialysis or an ultrafiltration procedure. Other such procedures which may cause hypotension or a symptom thereof in a patient would be readily recognized by persons of ordinary skill in the art as being within the scope of the invention

If desired, at the conclusion of a procedure that may induce hypotension in a patient, e.g., dialysis, a patient may be given one or more additional doses of midodrine in order to avoid hypotension which may occur post-dialysis. The dose for preventing post-dialysis hypotension is from 0.01 to 1 mg/kg of body weight administered as described above. The method of the subject invention can also be practiced by administering a particular salt, a derivative, an isomer, or a metabolite of midodrine, or a combination of any one or more of midodrine, or its salts, derivatives, isomers, or metabolites. The salt, derivative, isomer or metabolite of midodrine may be administered in the same dosage form, administration route, or dosage strength as disclosed herein for midodrine.

### DESCRIPTION OF PREFERRED EMBODIMENTS

### Example 1

A patient with no prior history of dialysis hypotension is dialyzed on a Baxter 550 volumetric machine with a bicarbonate bath with a concentration of 35 mEq/l, sodium 138 mEq/l, calcium 2.5 mEq/l, potassium 2.0 mEq/l and magnesium 1.5 mEq/l. When the patient exhibits objective symptoms of hypotension requiring medical intervention, an initial dose of 7.5 mg of midodrine is administered IV. Additional doses of midodrine may be administered depending on the patient's response to the initial dose.

### Example 2

A 60 year old patient with end-stage renal disease is scheduled for hemodialysis at 11 AM. The patient's resting blood pressure is 130/80 mm Hg. At 10:40 AM the day of the 11 AM hemodialysis, the patient swallows a tablet comprising 16 mg of desglymidodrine (approximately equal in activity to 20 mg of midodrine). Although the patient regularly develops adverse symptoms and hypotension during the dialysis session, the patient's blood pressure stays stable and no adverse symptoms are exhibited by the patient during hemodialysis.

In a subsequent dialysis day, the patient swallows a desglymidodrine 16 mg. tablet 20 minutes before the onset of dialysis. Despite this treatment, the patient develops hypotension one hour into the dialysis session. Intravenous desglymidodrine is then promptly administered by a nurse by a push infusion at a dose of 8 mg. The patient's blood pressure normalizes.

It is recognized that related embodiments not expressly provided herein are within the spirit of this disclosure. No omission in the current disclosure is intended to limit the current claims or disclosures. Certain preferred and alternative embodiments of the invention have been set forth for purposes of disclosing the invention; however, modifications to the disclosed embodiments may occur to those who are skilled in the art and those modifications are intended to be within the metes and bounds of the invention.

1. A method for the treatment of a patient receiving dialysis procedure, said method comprising:
administering to the patient during dialysis an effective amount of an anti-hypotensive agent to prevent or treat a complication of said dialysis procedure.

2. The method of claim 1, wherein said anti-hypotensive agent is a vasoconstrictive agent.

3. The method of claim 1 wherein said anti-hypotensive agent is an alpha-adrenergic agonist selected from the group consisting of midodrine, desglymidodrine, epinephrine, norepinephrine, dobutamine, phenylephrine, albuterol, terbutaline, amphetamine, tyramine, methylnorepinephrine, ephedrine, and phenylpropanolamine, or a salt, derivative, isomer, polymorph, or metabolite thereof.

4. The method of claim 3 wherein said alpha-adrenergic agonist is midodrine.

5. The method of claim 3 wherein said alpha-adrenergic agonist is desglymidodrine.

6. The method of claim 1 wherein the dialysis complication is selected from the group consisting of hypotension, nausea, vomiting, cramping, faintness, dizziness, weakness, fatigue, and diaphoresis.

7. The method of claim 1 wherein said treatment comprises administering a first dose of the anti-hypotensive agent to the patient during dialysis without a dose of said agent being administered within two hours before the onset of dialysis.

8. The method of claim 1, wherein the dialysis complication is symptomatic or intra-dialytic hypotension.

9. The method of claim 1 wherein said treatment further comprises administering a dose of the anti-hypotensive agent to the patient that follows a dose of said agent that was given either during the dialysis procedure or within two hours before the start of said procedure.

10. The method of claim 9, wherein said following dose of the anti-hypotensive agent is administered to the patient within about thirty minutes before completion of dialysis.

11. The method of claim 1 wherein said anti-hypotensive agent is administered orally.

12. The method of claim 1 wherein said anti-hypotensive agent is administered parenterally, including but not limited to intravenous, intramuscular, or subcutaneous administration.

13. The method of claim 1 wherein said anti-hypotensive agent is administered at a dose of 0.01 to 1 mg/kg of body weight of the patient.

14. The method of claim 1 wherein said anti-hypotensive agent is administered in response to a decrease in blood pressure.

15. The method of claim 1 wherein said method comprises administering the anti-hypotensive agent to the patient during dialysis to prevent post-dialytic hypotension or a symptom associated therewith.

16. The method of claim 1 wherein said anti-hypotensive agent is administered in combination with a second active pharmaceutical ingredient.

17. The method of claim 16 wherein said first anti-hypotensive agent and said second active pharmaceutical ingredient are administered sequentially.

18. The method of claim 16 wherein said first anti-hypotensive agent and said second active pharmaceutical ingredient are administered concomitantly or as part of a single dosage form.

19. The method of claim 1 wherein the patient suffers from acute renal failure.

20. The method of claim 19 wherein said acute renal failure is caused by an ingestion of a drug or a poison.

21. A method of preventing or treating a complication of dialysis in a patient in need of dialysis, said method comprising:
administering to the patient an effective amount of an anti-hypotensive agent selected from the group consisting of desglymidodrine, epinephrine, norepinephrine, dobutamine, phenylephrine, albuterol, terbutaline, amphetamine, tyramine, methylnorepinephrine, ephedrine, and phenylpropanolamine, or a salt, derivative, isomer, polymorph, or metabolite thereof.

22. The method of claim 21, said method comprising administering the anti-hypotensive agent within about ninety minutes before the onset of the dialysis procedure.

23. The method of claim 12 wherein said anti-hypotensive agent is administered as a continuous intravenous infusion.

24. The method of claim 21 wherein said administration is parenteral.

25. The method of claim 24, said method comprising administration of an anti-hypotensive agent selected from the group further consisting of midodrine.

26. The method of claim 21 wherein said patient lacks a condition or diagnosis of recurrent symptomatic or intradialytic hypotension.

27. The method of claim 26, said method comprising administration of an anti-hypotensive agent selected from the group further consisting of midodrine.

28. The method of claim 21 wherein said patient lacks a condition or diagnosis of renal failure.

29. The method of claim 21 wherein said patient suffers from acute renal failure.

30. The method of claim 29 wherein the acute renal failure is caused from ingestion of a drug or a poison.

31. The method of claim 28, said method comprising administration of an anti-hypotensive agent selected from the group further consisting of midodrine.

32. The method of claim 21 wherein the dialysis is not hemodialysis.

33. The method of claim 32, said method comprising administration of an anti-hypotensive agent selected from the group further consisting of midodrine.

34. The method of claim 1 wherein said dialysis is selected from the group consisting of hemodialysis, peritoneal dialysis, and ultrafiltration.

35. The method of claim 21 wherein said dialysis is selected from the group consisting of hemodialysis, peritoneal dialysis, and ultrafiltration.
